(19) United States
(12) Patent Application Publication
(10) Pub. No.: US 2006/0134077 A1
(43) Pub. Date: Jun. 22, 2006

(54) Electronic commerce system including customized catalog having encoded information

(76) Inventors: Bruce Verchere, British Columbia (CA); Rusung Tan, British Columbia (CA); Peter Liston, Ontario (CA); Robert G Korneluk, Ontario (CA)

Correspondence Address: CLARK & ELBING LLP, 101 FEDERAL STREET, BOSTON, MA, 02110, US

(21) Appl. No.: 10/502,283

(22) PCT Filed: Jan. 9, 2003
(86) PCT No.: PCT/US03/00597

§ 371 Date: Aug. 1, 2005

Related U.S. Application Data
(63) Continuation of application No. 10/051,263, filed on Jan. 18, 2002.

(51) Int. Cl. A61K 48/00; C12N 15/86

(52) U.S. Cl. 424/93.21; 435/456

(57) ABSTRACT

The invention features methods for the treatment of diabetes by administering a IAP nucleic acid sequence to a subject. One method includes the steps of (a) providing one or more insulin-producing cells or one or more precursor cells capable of producing progeny cells that produce insulin; (b) transducing the cells with a nucleic acid sequence encoding an IAP polypeptide, the nucleic acid sequence positioned for expression of the IAP polypeptide in the cells, wherein expression of the IAP polypeptide increases survival of the cells relative to untreated control cells; and (c)transplanting the cells from step (b) that have been transduced with a nucleic acid sequence encoding an IAP polypeptide into the individual, wherein the cells produce insulin in amounts sufficient to treat diabetes in the individual.

### BACKGROUND OF THE INVENTION

The invention relates to the treatment of diabetes.

Pancreatic islet cells are neuroendocrine organs that control blood glucose homeostasis. The precise interplay of a heterogeneous group of cell populations (beta, alpha, delta, and PP cells) results in the finely-tuned release of counterbalanced hormones (i.e., insulin, glucagon, somatostatin, and pancreatic polypeptide, respectively). Cytokines released by cells of the immune system that infiltrate the pancreatic islet cells, as well as cytotoxic T cell-mediated killing via the Fas/Fas-ligand and/or perforin/granzyme B pathways, however, can directly suppress and kill beta islet cells of the pancreas.

Beta islet cell apoptosis induced by a variety of immune mediators is central to the pathogenesis of autoimmune type 1 diabetes and the failure of islet cell transplantation. Loss of normal beta islet cell function has been implicated in type 2 diabetes, as well. Current therapy for type 1 diabetes generally involves injection of insulin in conjunction with management by diet and administration of drugs for the prophylaxis of cardiovascular disease. Type 2 diabetes can often be managed by diet, exercise, and medication. Whole pancreas transplantation has been successful for the treatment of both type 1 and type 2 diabetes, but the procedure is complex. Both pancreas and islet cell transplantation procedures are limited by the tremendous shortage of cadaveric pancreases suitable for transplantation. Beta islet cells from human or non-human sources (i.e., xenogeneic) are possible sources of cells for transplantation strategies, and pigs, in particular, are being studied as a potential source. Stem cells that have the ability to differentiate into beta islet cells are also being studied for use in transplantation to treat diabetes.

While current techniques for transplantation of beta islet cells show promise, the treatment results in only a 25% survival rate of the transplanted cells or tissue; fifty percent of the transplantable cells die prior to transplantation and another 25% die due to the recipient immune response.

There exists a need for therapies that increase the survival rate of insulin a producing cells used for transplantation.

### SUMMARY OF THE INVENTION

We have discovered that mice transplanted with beta islet cells expressing the human X-linked inhibitor of apoptosis protein (XIAP) show improved short and long-term survival following transplantation under the kidney capsule, relative to control mice. The number of transplanted cells is greatly increased even weeks following transplantation when the cells express XIAP. Based on our finding, we conclude that XIAP and related IAP polypeptides and polynucleotides, as well as related anti-apoptotic agents, can be successfully used in cell therapy protocols for the treatment of diabetes.

Accordingly, in a first aspect, the invention features a method of treating diabetes in an individual in need thereof The method includes the steps of: a) providing one or more cells capable of producing insulin or one or more precursor cells capable of producing progeny cells that produce insulin; b) transducing the cells with a nucleic acid sequence encoding an IAP polypeptide, the nucleic acid sequence positioned for expression of the IAP polypeptide in the cells, wherein expression of the IAP polypeptide increases survival of the cells relative to untreated control cells; and c) transplanting the cells from step b), wherein the transplanting results in the production of insulin by the cells in amounts sufficient to treat diabetes in the individual.

In a second, related aspect, the invention features a method of treating diabetes in an individual in need thereof. This method includes the steps of: (a) providing a cell capable of producing insulin or a precursor cell capable of producing progeny cells that produce insulin, in which the cell expresses a heterologous nucleic acid sequence encoding an IAP polypeptide, the nucleic acid sequence positioned for expression of the IAP polypeptide in the cell, wherein the IAP polypeptide is capable of inhibiting apoptosis of the cell; and (b) transplanting the cell into the individual, wherein the cell produces insulin in an amount sufficient to treat diabetes in the individual.

In various embodiments of the first or second aspect, the cell is transduced in situ, in vivo, or ex vivo; the cell is transduced with a viral vector (e.g., an adenoviral vector, an adeno-associated viral vector, a lentiviral vector, or a herpes viral vector); and the nucleic acid sequence is operably linked to a constitutive or inducible promoter that directs expression of the IAP polypeptide in the cell. Constitutive promoters include, for example, the CMV promoter, the SV-40 promoter, and the actin promoter. An example of an inducible or cell specific promoter is the insulin promoter (e.g., the human insulin promoter, the rat insulin promoter, or the bovine insulin promoter).

The IAP polypeptide can be, for example, XIAP, HIAP-1, HIAP-2, NAIP, survivin, livin, or a BIR domain-containing polypeptide having caspase-inhibiting activity (e.g., a polypeptide containing the BER3 domain from human XIAP). The IAP polypeptide can also be a component of a fusion protein.

In an embodiment of the first or second aspect of the invention, the cell can be contacted with an anti-apoptotic agent prior to and/or post transduction or transplantation in an amount sufficient to inhibit apoptosis of the cell.

In a third, related aspect, the invention features a method of treating diabetes in an individual in need thereof. This method includes the steps of: (a) providing a cell capable of producing insulin or a precursor cell capable of producing progeny cells that produce insulin; (b) contacting the cell with an anti-apoptotic agent in an amount sufficient to inhibit apoptosis of the cell; (c) transplanting the cell into the individual, wherein the cell produces insulin in an amount sufficient to treat diabetes in the individual.

In various embodiments of the first, second, or third aspect, suitable cells for transduction and/or transplantation include, for example, beta islet cells from a living donor or a cadaveric donor, stem cells (e.g., adult stem cells, embryonic stem cells) capable of differentiating into insulin-producing cells, and xenogeneic cells (e.g., cells from pig, sheep, or baboon). In one example, the cells are from a transgenic animal (e.g., a transgenic pig expressing XIAP in its beta islet cells).

The cells, whether transduced or treated with an anti-apoptotic agent, can be transplanted to a location in the subject that allows for engraftment and survival of the transplant, as well as delivery to the subject of insulin secreted by the insulin-producing cells. Suitable locations for the transplant include the pancreas, the liver (e.g., the liver portal vein), and the kidney (e.g., the kidney capsule). If desirable, the cells can be encapsulated prior to transplantation, as is described herein.

Desirably, the increase in survival of the transplanted cells is at least 20%, relative to control cells, and may be more than 50%, 75%, 100%, 200%, or 500%. Comparisons in the survival rate of cells can be made at any time following transplantation (e.g., one week, one month, three months, six months, one year, five years, etc.), but it is most desirable to make the comparison at one month following the transplant. It is also desirable that the transplanted cells survive in the recipient for more than a week; desirably three months; more desirably one year; and most desirably for the natural life of the individual receiving treatment.

Similarly, the cells, whether transduced or treated with an anti-apoptotic agent, will desirably produce insulin in amounts sufficient to treat diabetes in the recipient host for a longer period than will control cells. In certain embodiments, the cells will produce insulin in amounts sufficient to treat diabetes in the recipient for more than a week; desirably three months; more desirably one year; and most desirably for the natural life of the recipient.

In a further embodiment of the first, second, or third aspect, the method further includes the use of an immunosuppressive agent Examples of immunosuppressive agents include cyclosporin, cyclophosphamide, prednisone, dexamethasone, methotrexate, azathioprine, mycophenolate, thalidomide, FK-506, sirolimus, tacrolimus, daclizumab, and systemic steroids.

In a fourth aspect, the invention features a method of treating or preventing diabetes in an individual. In this method, the individual's beta islet cells are transduced with a nucleic acid sequence encoding an IAP polypeptide, such that the nucleic acid sequence is positioned for expression of the IAP polypeptide in the cells and expression of the IAP polypeptide increases survival of the cells, relative to untreated control cells, thus treating or preventing diabetes. In an embodiment of the fourth aspect, the cells can be transduced in situ or the cells can be removed from the individual, transduced ex vivo, and subsequently transplanted into the individual. In particular embodiments of the method of the fourth aspect, the cells are transduced, for example, with a viral vector (e.g., an adenoviral vector, an adeno-associated viral vector, a lentiviral vector, or a herpes viral vector); and the nucleic acid sequence is operably linked to a constitutive or inducible promoter that directs expression of the IAP polypeptide in the cell. As is stated for the methods of the first, second, and third aspects, the IAP polypeptide of the fourth aspect can be, for example, XIAP, HIAP-1, HIAP-2, NAIP, survivin, livin, or a BIR domain-containing polypeptide having caspase-inhibiting activity (e.g., a polypeptide containing the BIR3 domain from human XIAP), or can be a component of a fusion protein. In another embodiment of the fourth aspect of the invention, expression of the IAP polypeptide enhances the survival of the cell for one week; desirably three months; more desirably one year; and most desirably for the natural life of the individual receiving treatment.

In a fifth aspect, the invention features a method of treating diabetes in an individual in need thereof. This method includes the steps of: a) providing a cadaveric beta islet cell capable of producing insulin; b) transducing the cell with a recombinant adenovirus that includes a heterologous nucleic acid sequence encoding a human XIAP polypeptide, in which the nucleic acid sequence is positioned for expression in the cell, and the expression of the IAP polypeptide increases survival of the cell, relative to an untreated control cell, by inhibiting apoptosis of the cell; and c) transplanting the transduced cell into the individual, such that the transduced cell produces insulin in an amount sufficient to treat diabetes in the individual.

By “allogeneic” is meant genetically different members of the same species.

By “anti-apoptotic agent” is meant a polypeptide, a polynucleotide, a small molecule inhibitor, or any other compound that may be designed through rational drug design starting from known inhibitors of apoptosis that inhibit apoptosis. Examples of anti-apoptotic agents include the following: an IAP polypeptide (e.g., NAIP, HIAP1, HIAP2, Survivin, Livin, Appollon, TS-IAP/HILP2/AP, a BIR domain-containing polypeptide having caspase-inhibiting activity (e.g., a polypeptide containing the BIR3 domain from human XIAP), fusion proteins or chimeric proteins containing all or a fragment of a BIR domain-containing polypeptide having caspase-inhibiting activity, mammalian or viral orthologs or homologs of an IAP polypeptide, or any other IAP polypeptide described herein); caspase inhibitors (e.g., viral caspase inhibitors (e.g., baculovirus p35 or p49 protein or the polypeptide product of the CrmA Cowpox gene)); dominant negative caspase constructs (e.g., FLIP (natural caspase-8 dominant negative) or artificial dominant negative polypeptides of caspase-8, -9, -3, or -7); cell permeable peptidic caspase inhibitors (e.g., z-VAD-FMK, Boc-D-FMK Boc-Asp-FMK/BAF); non-peptidic, cell permeable, small molecule caspase inhibitors (e.g., pan-specific or mono-specific); small molecule caspase inhibitors (e.g., IDN-6556, IDN-6734, IDN-1965, IDN-5370, VX-740/pracnacasan, VX-799, MX-1013/CV-1013, L-826, 791, TBC-4521, anilinoquinazolines (e.g., AQZ-3 or AQZ-6), LDP-341, peptidomimetic caspase inhibitors (e.g., ketone- and aldehyde-containing derivatives of peptide caspase substrates); or any of the caspase inhibitors described in, e.g., U.S. Application Nos. 20020132833; 20020028803; 20020045623; 20020058630; 20020058631; 20020061853; 20020086832; 20020106404; 20020132833; 20020165230; 20020169177; or described in, e.g., U.S. Pat. Nos. 6,184,210; 6,201,118; 6,225,288; 6,355,618; or 6,495,522.

By “BIR domain” is meant a chain of amino acids that has at least 80%; desirably 85%; more desirably 95%; and most desirably 99% sequence identity to a sequence selected from the group consisting of amino acids 26-93, 163-230, or 265-330 of human XIAP, amino acids 26-93, 163-230, or 264-329 of mouse XIAP, amino acids 29-96, 169-235, or 255-322 of human HIAP-1, amino acids 29-96, 169-235, or 255-322 of mouse HIAP-1, amino acids 46-113, 184-250, or 269-336 of human HIAP-2, or amino acids 25-92, 156-222, or 241-308 of mouse HIAP-2.

By “caspases” is meant cysteine proteases (named for a cysteine residue in the active site) that cleave their substrates after specific aspartic acid residues (Asp-X) that are produced from inactive zymogens known as procaspases. Procaspases require proteolytic cleavage at specific internal aspartate residues for activation. The procaspase is arranged such that the N-terminal peptide (prodomain) precedes a large subunit domain, which precedes a small subunit domain. The large subunit contains the conserved active site pentapeptide QACXG (X═R, Q, G) that contains the nucleophilic cysteine residue. The small subunit contains residues that bind the Asp carboxylate side chain and others that determine substrate specificity. The human caspase family includes Ced-3, human ICE (interleukin-1-β converting enzyme) (caspase-1), ICH-1 (caspase-2), CPP32 (caspase-3), caspase-4, caspase-5, Mch2 (caspase-6), ICE-LAP3 (caspase-7), Mch5 (caspase-8), ICE-LAP6 (caspase-9), Mch4 (caspase-10), caspase 11-14, and others (see, e.g., Martin and Green, Cell 82:349-352 (1995); Thomberry, Chem Biol., 5:R97-R103 (1998)).

Two subfamilies of caspases are of central importance in the intracellular signaling pathways leading to apoptosis. One subfamily consists of the enzymes involved in initiating events in the apoptotic pathway, including transduction of signals from the plasma membrane. Members of this subfamily include caspases-2, 8, 9 and 10. The other subfamily, consisting of the effector capsases 3, 6, and 7, are involved in the final downstream cleavage events that result in the systematic breakdown and death of the cell by apoptosis.

By “caspase-inhibiting activity” is meant the inhibition of a caspase by more than 10%, 20%, 30%, 50%, 60%, 70%, 80%, 90%, 95%, or 99%, as determined by the methods disclosed in U.S. Pat. No. 6,228,603.

By “a cell capable of producing insulin” is meant a cell that is capable of producing insulin when transplanted into a suitable environment (e.g., transplanted into the liver, the kidney, or the pancreas) in a recipient. The cell may or may not be producing insulin at the time of transduction or transplantation. For example, the cell can be a stem cell that can differentiate into a cell that produces insulin. Differentiation can occur either prior to or following transplantation into the recipient. The cell can also be genetically modified to produce insulin by methods known in the art.

By “cells recombinantly engineered for transplantation into humans” is meant that the cells (e.g., pig, sheep, or baboon cells) are engineered to lack expression of antigens that would otherwise activate an immune response. For example, cells derived from an animal other than the recipient that are recombinantly engineered to lack the alpha-galactosyl xenoepitope are an example of such a cell and result in avoidance of hyperacute rejection mediated by preexisting antibodies to that epitope.

By “heterologous IAP nucleic acid” is meant that the LIP-encoding nucleic acid sequence referred to is derived from a species other than the species of the cell into which the nucleic acid sequence is introduced.

By “IAP polypeptide” is meant a polypeptide having at least one BIR domain and caspase-inhibiting activity (thus being capable of inhibiting apoptosis of a cell). In desired embodiments the IAP polypeptide is a polypeptide having at least 80%; desirably 90%; more desirably 95%; and most desirably 99% or greater amino acid sequence identity to at least one of the IAP amino acid encoding sequences of FIGS. 1-4 of U.S. Pat. No. 6,156,535, or a BIR-containing portion thereof (i.e., the BIR domains from the human or murine xiap, hiap-1, and hiap-2). Desirably, the region of sequence over which identity is measured is a region encoding at least one BIR domain.

The term “IAP polypeptide” also encompasses naturally-occurring mammalian homologs, including, e.g., allelic variants, natural mutants, induced mutants, proteins encoded by a nucleic acid sequence that hybridizes to the complement of IAP nucleic acid sequences that encode the amino acid sequences described above under high stringency conditions or low stringency conditions (e~g., washing at 2×SSC at 40° C. with a probe length of at least 40 nucleotides), and polypeptides or proteins specifically bound by antisera directed to an IAP polypeptide. The term also includes chimeric polypeptides that comprise an IAP fragment, e.g., a portion of a naturally-occurring IAP polypeptide or homolog, as long as the fragment or chimeric polypeptide is capable of inhibiting apoptosis of a cell. A chimeric polypeptide can contain, e.g., one BIR domain (e.g., BIR1 from human XIAP (Genbank Accession No. AAC50518.1)) or a second BIR domain from another species (e.g., BIR2 from mouse IAP (Genbank Accession No. AAH11338)).

By “IAF nucleic acid” is meant a nucleic acid sequence encoding an IAP polypeptide.

By “immunoprivileged” is meant a cell that does not elicit an immune response when transplanted into a particular individual. By “immune response” is meant a response made by the immune system of an individual to a foreign substance. An immune response, as used herein, includes events that lead to transplant or graft rejection, antibody production, inflammation, or the response of antigen-specific lymphocytes to antigen. An immune response may be detected, for example, by determining if transplanted material has been successfully engrafted or has been rejected.

By “immunosuppression” is meant prevention or delay of the immune response in the recipient and can be accomplished by treating a recipient with an immunosuppressive agent which prevents or delays the occurrence of, or decreases the intensity of, an immune response in the recipient, as compared to a transplant recipient that has not received an imnmunosuppresive agent. A delay in the occurrence of an immune response can be a short delay, for example, a delay of one day, or a long delay, for example, a delay of more than thirty days.

By “immunosuppressive agent” is meant any agent that prevents, delays the occurrence of, or reduces the intensity of an immune reaction against a foreign cell in a recipient host, particularly a transplanted cell. Examples of immunosuppressive agents include, but are not limited to, cyclosporin, cyclophosphamide, prednisone, dexamethasone, methotrexate, azathioprine, mycophenolate, thalidomide, FK-506, sirolimus, tacrolimus, daclizumab, and systemic steroids.

By an “inmmunotolerant individual” is meant a recipient host that fails to mount an immune response to a given foreign substance. A recipient host that is “inmunotolerant” does not reject or destroy transplanted material to the extent that an immune responsive recipient host does and does not respond to a given foreign substance (i.e., an antigen) by producing antibodies capable of binding to the antigen. Transplanted or grafted material can be rejected by the immune system of the recipient host unless the recipient host is immunotolerant to the transplanted material or immunosupressive drugs are used to prevent rejection.

By “increases survival” is meant that transduction of a cell (e.g., beta islet cells) with a nucleic acid sequence encoding an IAP polypeptide (e.g., XIAP) reduces the likelihood of apoptosis in the cell and promotes an increase in the lifespan of the cell. An increase in survival is desirably an increase in cell longevity by 2 fold; desirably 10 fold; more desirably 50 fold; and most desirably by 500 fold or more as compared to the longevity of an untreated control cell.

By “inhibiting apoptosis” is meant decreasing the number of cells that die by means of apoptosis, relative to an untreated control. The decrease is by at least 25%; desirably by at least 50%; more desirably by at least 75%; and most desirably by at least one-fold.

By “isogeneic” is meant having an identical genetic constitution.

By “modulating apoptosis” is meant increasing or decreasing the number of cells that would otherwise undergo apoptosis in a given cell population. Desirably, the cell population is selected from a group including beta islet cells or any insulin-producing cells (either naturally-occurring or engineered by recombinant techniques). It will be appreciated that the degree of modulation provided by an IAP polypeptide in a given assay will vary, but that one skilled in the art can determine the statistically significant change in the level of apoptosis modulated by an IAP polypeptide.

By a “nucleic acid sequence” is meant a chain of nucleotides that may be covalently linked to other nucleotides.

By “operably linked” is meant that a first nucleic acid sequence and one or more regulatory sequences are connected in-such a way as to permit expression of the first nucleic acid sequence under the appropriate conditions (e.g., in the presence of transcriptional activator proteins, polymerases, or elongation factors).

By “polypeptide” is meant any chain of more than two amino acids, regardless of post-translational modification such as glycosylation or phosphorylation.

By “pancreatic stem cell” is meant a stem cell that has been isolated from pancreatic tissue and/or a stem cell that has the ability to differentiate into a pancreatic cell (e.g., a beta islet cell).

Reference to a nucleic acid sequence “positioned for expression” means that a first nucleic acid sequence encoding a polypeptide is positioned proximal to a second nucleic acid sequence that directs transcription and translation of the first nucleic acid sequence (i.e., facilitates the production of, e.g., an IAP polypeptide). The second nucleic acid sequence may be one that naturally occurs proximal to the first nucleic acid sequence encoding the polypeptide or may be a nucleic acid sequence introduced by artifice.

By “promoter” is meant a nucleic acid sequence that is sufficient to direct transcription of an operably-linked nucleic acid sequence that encodes a polypeptide.

By “pseudo-islet cell” is meant a cell, other than a beta islet cell, which does not normally express insulin in a glucose-stimulated manner, and which has been genetically modified to produce insulin by, for example, insertion of a nucleic acid sequence encoding an insulin gene, such that the cell produces insulin.

By “regulatory sequence” is meant a region of a nucleic acid to which regulatory molecules, such as repressors or enhancers, bind, thereby altering the expression of the adjacent gene.

By “rejection” is meant a strong immune response to transplanted material by the immune system of the recipient. Specifically, rejection means death of more than 90% of the transplanted cells or tissue as a consequence of the immune response of the recipient. The occurrence of transplant rejection and/or the rate at which rejection occurs following transplantation may vary depending on factors that include the nature of the transplanted material (i.e., the cell type, or the cell number) and the recipient (i.e., whether or not the recipient is immunotolerant, has been treated with an immunosuppressive agent, or both).

By “stem cell” is meant an undifferentiated cell that is capable of essentially unlimited propagation under appropriate conditions in vivo or ex vivo and is capable of differentiation to other cell types (e.g., a progenitor or precursor cell, such as a pancreatic precursor cell, or a fully differentiated cell, such as a pancreatic beta islet cell). Stem cells include, for example, embryonic stem cells and adult stem cells.

By “substantially identical” is meant a polypeptide or nucleic acid exhibiting at least 50%; desirably 85%; more desirably 90%; and most desirably 95% homology to a reference amino acid or nucleic acid sequence. For polypeptides, the length of comparison sequences will generally be at least 16 amino acids; desirably at least 20 amino acids; more desirably at least 35 amino acids; and most desirably the full length of the reference sequence.

Sequence identity is typically measured using BLAST® (Basic Local Alignment Search Tool) or BLAST® 2with the default parameters specified therein (see, Altschul et al., J. Mol. Biol. 215:403410 (1990); and Tatiana et al., FEMS Microbiol. Lett. 174:247-250 (1999)). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “transduced cell” is meant a cell into which (or into an ancestor of which) has been introduced, by means of recombinant molecular biology techniques, a nucleic acid sequence encoding an IAP polypeptide.

By “transduction” is meant any method for introducing foreign molecules into a cell. Lipofection, calcium phosphate precipitation, viral delivery (such as by a vector consisting of an adenovirus, adeno-associated virus, a lentivirus, a herpes virus, or a retrovirus, for example), electroporation, and biolistic transformation are just a few of the methods that may be used.

By “transgene” is meant any piece of a nucleic acid sequence that is inserted by artifice into a cell, and becomes a stable component of the organism which develops from that cell. Such a transgene may include a nucleic acid sequence that is partly or entirely heterologous (i.e., foreign) to the recipient host cell or organism, or may represent a nucleic acid sequence homologous or identical to an endogenous gene of the organism.

By “transgenic” is meant any cell that includes a DNA sequence which is inserted by artifice into a cell and becomes part of the genome of the organism which develops from that cell. As used herein, the transgenic organisms are generally transgenic mammals (e.g., rodents, such as rats or mice) and the nucleic acid sequence (transgene) is inserted by artifice, desirably into the nuclear genome.

By “transplanting” or “transplantation” is meant the grafting of cells or tissue into a recipient. The transplanted cells or tissue can be allogeneic, isogeneic or xenogeneic.

By “treating diabetes” is meant that an individual previously diagnosed with diabetes requires 10%; desirably 50%; more desirably 75%; and most desirably 90% less insulin per day.

By an “untreated control cell” is meant a cell (e.g., a beta islet cell) that has not been transduced with an expression vector comprising a nucleic acid sequence encoding an IAP polypeptide, or a cell that has not been exposed to an anti-apoptotic agent, relative to the cell being tested. Control cells may encode proteins other than an IAP polypeptide (e.g., a reporter protein such as green fluorescent protein or β-galactosidase).

By “xenogeneic” is meant from members of different species.

The present invention provides several advantages over current therapies. Beta islet cells are extremely fragile and isolation procedures typically result in a survival level of less than fifty percent upon completion of the isolation. Fifty percent of those cells isolated subsequently die before being transplanted into the recipient. Furthermore, less than twenty-five percent of the transplanted cells typically engraft stably in the recipient's body. Expression of XIAP, or a related IAP polypeptide, in a beta islet cell, or contact of beta islet cells with an anti-apoptotic agent, increases survival of the beta islet cells prior to and after transplantation by decreasing cell death at the various stages of the procedure. Once engrafted, the beta islet cells of the invention are protected from challenge by the immune system. Although immunosuppressive drugs have historically been used to prevent rejection of beta cell grafts, such drugs often impair the ability of the transplanted cells to respond to glucose and produce insulin. By increasing the survival of, and decreasing the extent of, the immune challenge to beta islet cells, expression of IAP polypeptides can greatly improve the outcome for the recipient.

Other features and advantages of the invention will be apparent from the following description of the preferred embodiments thereof, and from the claims.

### BRIEF DESCRIPTION OF THE DRAWINGS

FIGS. 1A and 1B are photographs of western blots containing protein extracted from MIN-6 (FIG. 1A) and NIT-1 (FIG. 1B) cells (pancreatic insulinoma beta islet cells) 48 hours post-infection. The protein was detected using an anti-human XIAP antibody. The position and size of the XIAP polypeptide, as indicated, is 57 kDa. Infecting MIN and NIT cells with increasing pfu of Ad-XIAP increases the amount of human XIAP polypeptide being expressed and indicates that XIAP can be overexpressed in beta islet cells

FIGS. 2A-2C are photographs of MIN-6 beta islet cells that have been untreated (FIG. 2A), treated with cytokines (TNF, IL-1, and IFN ) for 48 hours (FIG. 2B), or treated with a recombinant adenovirus containing an XIAP transgene (Ad-XIAP), followed by treatment with cytokines (FIG. 2C). Death of MIN-6 cells following cytokine exposure was 32±8% vs. 13±1% in untreated cells. XIAP expression reduced cytokine-mediated cell death in MIN-6 cells to 16±6% at 5 pfu/cell and 22±4% (10 pfu/cell). These data demonstrate that XIAP expression protects MIN-6 beta islet cells from cytokine-mediated death.

FIGS. 3A and 3B are graphs representing the Ad-XIAP-mediated protective effect on MIN-6 beta islet cells (FIG. 3A) and NIT-1 beta islet cells (FIG. 3B). Expression of XIAP reduces cytokine-mediated cell death.

FIG. 4 is a photograph of a western blot showing expression of XIAP in isolated BALB/c mouse islet cells by adenoviral transduction (Ad-XIAP) using 0, 5, or 10 pfu/cell.

FIG. 5 is a graph showing that the expression of XIAP does not impair beta islet cell function in vitro. Glucose-stimulated insulin secretion from non-transduced and Ad-XIAP transduced Balb/c islet cells was determined after 48 hours in culture. Islet cells were incubated in the presence of 1.67 or 16.7 mM glucose for 1 hour, followed by measurement of immunoreactive insulin in the incubation media. Insulin secretion was calculated as a percent of basal (low glucose).

FIG. 6 is a graph showing the blood glucose levels of the mice transduced with Ad-XIAP or Ad-LacZ. Prior to the streptozotocin injection, all mice are normoglycemic. Approximately three days post-injection, the mice become diabetic, at which time the mice are transplanted with the transduced beta islet cells. At 1-2 days post-transplantation, the mice become normoglycemic as a result of the transplant. Mice transplanted with Ad-XIAP transduced beta islet cells exhibit reduced blood glucose levels, as opposed to mice transplanted with Ad-LacZ (control) transduced cells. Therefore, expression of XIAP promotes a protective effect in islet cell allografts in diabetic mice.

FIG. 7 is a graph showing the survival profile of Ad-XIAP transduced islet allograft following transplantation into diabetic recipients. Ad-LacZ represents the control. Mice receiving XIAP expressing beta islet cells remain normoglycemic for greater than 60 days, while mice receiving the Ad-LacZ-treated beta islet cells become hyperglycemic after fewer than 25 days. The experiment was terminated so that the mice could undergo histological examination.

FIG. 8 is a graph depicting the average blood glucose levels of mice transplanted with Ad-XIAP transduced islet cells (n=6) and mice transplanted with control islet cells (n=7; i.e., islet cells transduced with an Ad-LacZ vector). The recipients of the Ad-XIAP remain normoglycemic until 56 days, at which point the surviving mice are nephrectomized and blood glucose levels are determined to verify that survival resulted from the transplanted islet graft. In contrast, the Ad-LacZ mice fail to remain normoglycemic beyond 5 days and become hyperglycemic after 14 days.

FIGS. 9A-9D are photographs showing the protective effect of XIAP expression in insulin-positive beta cells following allotransplantation into diabetic recipients. Immunostaining of insulin-positive beta cells (FIGS. 9A and 9C) and glucagon-positive alpha cells (FIGS. 9B and 9D) in islet grafts excised from an Ad-lacZ transplant that failed at day 10 post-transplantation (FIGS. 9A and 9B) or from an Ad-XIAP transplant at day 47 (FIGS. 9C-and 9D). Note the preservation of both alpha and beta cells in the XIAP expressing graft, but not the control (lacZ expressing) graft.

FIGS. 10A-10F are photographs showing the decrease in immune cell infiltration into transplanted islet allografts overexpressing XIAP. Immunostain for insulin and leukocyte common antigen showing insulin-positive beta cells (FIGS. 10A, 10C, and 10E) and leukocyte infiltration (FIGS. 10B, 10D, and 10F) in islet grafts from Ad-lacZ (FIGS. 10A and 10B) or Ad-XIAP (FIGS. 10C and 10D) grafts excised at day 10 post-transplantation, or an Ad-XIAP graft excised at day 60 post-transplantation (FIGS. 10E and 10F). Note the almost total loss of infiltrating immune cells (stained for leukocyte common antigen) in the XIAP expressing graft by day 60 post-transplantation.

### DETAILED DESCRIPTION

Beta islet cell apoptosis, induced by the immune response, is central to the pathogenesis of autoimmune (type 1) diabetes and the subsequent failure of islet transplants, where attempted. Loss of normal beta islet cell function has also been implicated in type 2 diabetes. Proinflammatory cytokines are thought to be important mediators of beta islet cell death in autoimmune diabetes and are thought to act at least in part via activation of caspases. Therefore, we hypothesized that expression of an IAP polypeptide in beta islet cells may protect them from cytokine-mediated death. The inhibitor of apoptosis polypeptides (IAP) inhibit apoptosis in many different cell types, likely via inhibition of caspase-3, caspase-9, or other initiator and executioner caspases. We have discovered that by increasing expression of an IAP polypeptide in beta islet cells, we can improve protection from immune-mediated cell death in vivo, in vitro, and including following transplantation.

Here we provide methods for the treatment of diabetes in which IAP polynucleotides, polypeptides, or anti-apoptotic agents are provided to beta islet cells in a diabetic patient to prevent apoptosis of these cells. Our results provide a therapy for the effective treatment of diabetes.

IAP Therapy

Based on our discovery that Ad-XIAP increases the survival of transplanted beta islet cells in a mouse model for diabetes and improves the glucose levels in these mice, we believe that XIAP and other IAP polynucleotides, and polypeptides can be used in ex vivo and in in vivo gene therapy methods. In particular, to increase the survival of beta islet cells or other insulin-producing cells, we provide methods to introduce a nucleic acid encoding an IAP polypeptide into the cells, followed by transplantation of the cells into a diabetic individual. Entry of the nucleic acid sequence is accomplished using suitable techniques known in the art.

In a method of the invention, an IAP nucleic acid sequence is operably linked to a promoter (e.g., a constitutive promoter or an inducible promoter) and contained in an expression vector, such as a viral vector (for example, an adenoviral vector, an adeno-associated viral vector, a lentiviral vector, a herpes viral vector, or a retroviral vector). Numerous vectors useful for this purpose are generally known (Giannoukakis et al., Diabetes 48:2107-2121 (1999); Efrat, Eur. J. of Endocrinol. 138:129-133 (1998); Stone et al., J. Endocinol 164:103-118 (2000); Castro et al., Baillieres Best Prac. Res. Clin. Endocrinzol. Metab. 13:431-449 (1999); Becker et al., J. Biol. Chem. 59:21234-21238 (1994); Csete et al., Transplantation 59:263-268 (1995); Sigalla et al., Hum. Gen. Ther. 8:1625-1634 (1997); Leibowitz et al., Diabetes 48:745-753 (1999); Guo et al., Cell Transplant 8:661-671 (1999).

Non-viral approaches may also be employed for the introduction of therapeutic IAP nucleic acid sequences into cells which are otherwise likely to undergo apoptosis. For example, an IAP polypeptide may be introduced into a beta islet cell, or any other cell capable of producing insulin (such as those disclosed below), by the technique of lipofection, asialorosonucoid-polylysine conjugation, calcium phosphate coprecipitation, electroporation, or biolistic transfection, all of which have been optimized in pancreatic islet cells (for review see Levine et al., Diabetes Metab. Rev. 13:209-246 (1997)).

In the gene therapy constructs, IAP polypeptide expression is directed from any suitable promoter (e.g., the human cytomegalovirus, simian virus 40, or metallothionein promoters, the insulin promoter (e.g., the human insulin promoter or rat insulin promoter), or the actin promoter (e.g., the chicken actin promoter)). The production of the IAP polypeptide can be further controlled by the use of appropriate manunalian regulatory elements. For example, if desired, enhancers known to direct preferential gene expression in beta islet cells, or another suitable cell type, may be used to direct IAP polypeptide expression. Such enhancers include, without limitation, those enhancers that are characterized as tissue or cell specific in their expression.

Any cell suitable for transplantation for the treatment of diabetes can be employed in the methods of the invention. In particular, pancreatic beta islet cells isolated from living donors (i.e., allogeneic or isogenic cells) or cadaveric donors can be used in the methods of the invention. Also useful are non-beta cells which have been selected or engineered to produce insulin in a glucose-regulated manner.

Partial pancreatectomies, which have been demonstrated to be effective in mice (see, for example, Hardikar et al., J. Endocrinol. 162:189-195 (1999)) can be performed to provide a source of beta islet cells for transplantation from living donors. The cells are removed, partially purified, and then utilized for transplantation. Whole pancreases isolated from cadavers can also be a source of beta islet cells for transplantation. The cells of the invention can be prepared using a suitable vector (as disclosed herein) to express an IAP polypeptide.

Another source of allogeneic or xenogeneic cells for transplantation are stem cells (i.e., adult stem cells and embryonic stem cells (ES) cells). Such stem cells can be isolated from donors and differentiated into beta islet cells and used in the methods of the invention. Desirable stem cells that can be used in the methods of the invention are described in Toma et al. (Nat. Cell Biol. 3:778-784 (2001)) and PCT Application No. WO 01/53461. Ramiya et al. have demonstrated that islet cells generated in vitro from pluripotent stem cells from the pancreatic ducts of adult prediabetic non-obese diabetic (NOD) mice differentiate to form glucose-responsive islet cells that are capable of reversing insulin-dependent diabetes. The cells are functional with or without encapsulation (Raniya et al., Nature Med., 6:278-282 (2000)). In addition, undifferentiated ES cells administered to streptozotocin-induced diabetic mice have been shown to normalize glycemia in those mice (J. Clin. Invest. 98:216-224 (1996); Li et al., Curr. Biol. 8:971-974 (1998)). The differentiation of ES cells for the generation of insulin-producing structures is described in Lumelsky et al. (Science 292:1389-1394 (2001)).

Stem cells, according to the invention, can be identified by their expression of nestin (as described in U.S. Application No. 20010046489 and in Zulewski et al., Diabetes 50:521-533 (2001)), which, in turn, can be detected using FACS, immunocytochemical staining, RT-PCR, Southern, northern, and western blot analysis, and other such techniques of cellular identification as known to one skilled in the art. Allogeneic or isogeneic stem cells can be isolated from a preparation of pancreatic tissue, for example, islet cells obtained from a biopsy sample of tissue from a diabetic patient. The cells can then be expanded ex vivo and the resulting progeny cells can be transplanted back into the donor. Inside the donor, such cells may differentiate to provide insulin-secreting cells, such as beta islet cells, to cells lost to the autoimmune attack that caused the diabetes. This approach can overcome the problem of immune rejection which might otherwise result from transplantation of beta islet cells from a different individual.

An advantage of the use of stem cells over differentiated islet cells is that stem cells may be more adaptive by virtue of their ability to differentiate in situ. Use of stem cells may result in improved microcirculation and production of the full complement of different islet cell types which, in turn, are more responsive to the physiological needs of the recipient.

Human long-term repopulating hematopoietic stem cells (LTR-HSCs) have also been shown to undergo significant ex vivo expansion (Nakahata, Int. J. Heinatol. 73:6-13 (2001)) and may provide a source of transplantable HSCs suitable for use in the methods of the invention.

Beta islet cell precursors are another source of cells for use in the methods of the invention because they can have an increased proliferative potential. In vivo, the neogenesis of endocrine islet cells from ductal epithelium precursor cells has been described after 90% pancreatectomy and pancreas wrapping in the rodent, or when transplanted together with fetal mesenchyme into nude mice (Bonner-Weir et al., Diabetes 42:1715-1720 (1993); Vinik et al., Tumour Biol. 14:184-200 (1993); Dudek et al., Diabetes 40:1041-1048 (1991)). In vitro, the neogenesis of endocrine islet cells for transplantation may be obtained from ducts using cells, matrix, and growth factors. These methods increase the mass of transplantable endocrine tissue which can be obtained from adult cadaveric pancreases (Kerr-Conte et al., Diabetes 45:1108-1114 (1996)). For example, it has been shown that human ductal cells exposed to MATRIGEL® and growth factors differentiate into islet endocrine cells in vitro (Bonner-Weir et al., PNAS USA 97:7999-8004 (2000)).

The generation of functional beta islet cell lines can overcome the shortage of tissue for transplantation without the need for complex matrices and growth factors to stimulate in vitro expansion. Such cell lines are a more reproducible source than newly harvested islet cells and can be more readily genetically modified to improve transplantation properties. Methods for obtaining such cells beta islet cell lines have been described (Knaack et al., Diabetes 43:1413-1417 (1994); Newgard, Diabetes 43:341-350 (1994); Asfari et al., Endocrinology 130:167-178 (1992); Poitout et al., Diabetes & Metabolism 22:7-14 (1996); Efrat, Diabetes Reviews 4:224-234 (1996)). The sources for the beta islet cell lines include spontaneous insulinomas, carcinogen-induced insulinomas, oncogenic virus-induced insulinomas, insulinomas induced by a combination of carcinogen treatment and oncogenic viruses, and insulinomas from transgenic mice expressing dominant oncogenes, particularly SV40 T antigen under the control of the insulin promoter (see, e.g., Rae et al., Can. J. Physiol. Pharmacol. 57:819-824 (1979); Chick et al., PNAS USA 74:628-632 (1977)); Santerre et al., PNAS USA 78:4339-4343 (1981)); Uchida et al., J. Natl. Cancer Inst. 63:119-126 (1979)); Santerre et al., PNAS USA 78:4339-4343 (1981)); Drucker, Am. J. Physiol. 267:E629-E635 (1994); Hanahan, Nature 315:33-40 (1985); Radvanyi et al., Mol. Cell. Biol. 13:4223-4232 (1993). The most studied pancreatic cell lines are those derived from a radiation-induced, transplantable insulinoma found in inbred New England Deaconess Hospital (NEDH) albino rats (Chick et al., PNAS USA 74:628-632 (1977)), including the RIN (Gazdar et al., PNAS USA 77:3519-3523 (1980), MSL (Masden et al., J. Cell Biol. 103:2025-2034 (1986)), INS (Asfari et al., Endocrinology 130:167-178 (1992)), and CRI (Carrington et al., J. Endocrinol. 109:193-200 (1986)) series of cell lines.

Another source of cells that may be used in the methods of the invention are neuroendocrine cells, such as those found in the pituitary and adrenal glands. These cells possess the secretory machinery needed for a regulated secretion of polypeptide hormones in response to external stimuli. Due to the expression of prohormone convertases (PCs) 2 and 3, some of these cells can process proinsulin to insulin without any further genetic manipulation and represent a potential source of cells for use in the methods of the invention. Many of the studies using neuroendocrine cells as a target for insulin gene therapy have been performed in AtT20, a neuroendocrine cell line derived from a mouse pituitary corticotroph tumor (see, e.g., Moore et al., Cell 35:531-538 (1983); and Furth et al., Proc. Soc. Exp. Biol. Med. 84:253-254 (1953)). AtT20 cells express PC2, PC3 and glucokinase (Hughes et al., J. Biol. Chem. 266:4521-4530 (1991)), but no GLUT-2. Transfection of the AtT20 cells with the proinsulin gene allowed the cells to secrete insulin (Hughes et al., PNAS USA 89:688-692 (1992); Irmger et al., J. Biol. Chem. 269:1756-1762 (1994); Kaufmann et al., Biochem. J. 310:869-874 (1994); Stewart et al., J. Mol. Endocrinol. 11:335-341 (1993)).

Hepatocytes also express elements of the glucose-sensing machinery such as glucokinase and GLUT2, and respond to extracellular glucose levels. These properties make them an attractive starting material for use in gene therapy protocols for the treatment of diabetes. To optimize the ability of the cells to efficiently convert proinsulin to insulin the endoproteases PC2 and PC3 were introduced into the cells and the proinsulin cleavage sites were modified to be furin-cleavable sites (Kaufmann et al., Diabetes 46:978-982 (1997); Vollenweider et al., Diabetes 44:1075-1080 (1995); Groskreutz et al., J. Biol. Chem. 269:6241-6245 (1994); Falqui et al., J. Mol. Med. 77:250-253 (1999); Muzzin et al., Mol. Endocrinol. 11:833-837 (1997)).

Beta islet cells from other sources, such as xenogeneic cells from pig, sheep, or baboon, can be used in the methods of the invention. Animals useful as a source of xenogeneic cells are those that can be bred quickly and are free of diseases affecting recipients. Xenogeneic cells for transplantation can be encapsulated to reduce the immune system response while allowing exchange of insulin, and waste for nutrients and oxygen. Xenogeneic cells can also be isolated from genetically engineered animal sources that lack antigens that elicit hyperacute rejection (i.e., the alpha-galactosyl xenoepitope in pigs or sheep; Denning et al., Nature Biotechnology 19:559-562 (2001)). In addition, these animal sources of xenogeneic cells can be engineered to express an IAP polypeptide (e.g., a XIAP polypeptide or a XIAP BIR3-containing polypeptide), thereby overcoming the need to provide a source of IAP polypeptide to the cells to be transplanted in a separate step.

Transplantation

Transplantation, according to the invention can include the steps of isolating a cell (e.g., a stem cell or a beta islet cell), transducing the cell with a nucleic acid sequence encoding an IAP polypeptide, and transferring the cell into a recipient. Transplantation according to the invention can involve transferring a cell into a recipient by injection of a cell suspension into the recipient, surgical implantation of a cell mass into a tissue or organ of the recipient (e.g., the liver, the kidney capsule, or the pancreas), or perfusion of a tissue or organ of the recipient with a cell suspension. The route of transferring the cell or transplantation, will be determined by the need for the cell to reside in a particular tissue or organ and by the ability of the cell to find and be retained by the desired target tissue or organ. In the case where a transplanted cell is to reside in a particular location, it can be surgically placed into a tissue or organ or be injected into the bloodstream (if the cell has the capability to migrate to the desired target organ).

Another transplantation method of the invention includes the additional step of culturing the cell to be transplanted prior to transferring the cell into a recipient.

In another method, the cell isolated is a stem cell and the method further includes differentiating the cell prior to or after transduction with a nucleic acid sequence encoding an IAP polypeptide before transfer of the cell into a recipient

As noted above, the donor cells or tissue used in the methods of the invention can be isolated from an allogeneic, isogeneic, or xenogeneic donor. In all cases, the expression of an IAP polypeptide in the cells promotes their survival and can be sufficient to prevent immune rejection of the cells by the recipient. In methods where the source material is derived from a donor with a different genetic background from that of the recipient, it may be desirable to administer an immunosuppressive agent to the recipient prior to and/or post transplantation to prevent immune rejection of the transplanted material and to supplement the survival-enhancing effect of the IAP polypeptide. It is not always necessary to suppress the immune system for an extended period of time. We have discovered that use of an immunosuppressive agent for as briefly as one week (one month, six months, or one year) may be sufficient to prevent immune rejection of the transplanted material. Administration terms of about a month, six months, or one year may also be used. If needed, an immunosuppressive agent can be used for an extended period of time (e.g., the life of the recipient) to further protect the transplanted cells from immune rejection.

An anti-apoptotic agent may be used with the methods of the invention to enhance the survival of cells in the transplantation protocols. Such agents include, e.g., a polypeptide, a polynucleotide, a small molecule inhibitor, or any other compound that may be designed through rational drug design starting from known inhibitors of apoptosis. Inhibitors of apoptosis can be administered as anti-apoptotic therapeutic agents by methods known to one skilled in the art.

IAP Polypeptides

Inhibitor of apoptosis proteins (IAPs) are proteins that have been actively studied in their role of modulating apoptosis via interaction with caspases. IAPs have been isolated from several sources, including humans (e.g., HIAP-1, HIAP-2, and XIAP) and it has been demonstrated that portions of naturally occurring IAPs are capable of acting as caspase inhibitors. For example, the BIR3 domain of XIAP exhibits caspase-9 inhibitory activity and may be used in the methods of the invention. The full-length IAPs, as well as caspase-inhibiting fragments and analogs (i.e., polypeptides having substantial identity to an IAP and exhibiting caspase-inhibiting activity) are collectively referred to as “IAP polypeptides.”

Exemplary IAPs, identified by their Genbank Accession Number, are as follows: P98170, AAC50518, XP_{—}013050, NP_{—}001158, S69544, AAG22969, NP_{—}0.71567, Q90660, Q62210, NP_{—}068520, NP_{—}076477, XP_{—}040715, AAK81892, AAL47170, AAC83232, AAL33679, NP_{—}001156, Q60989, AAB88044, NP_{—}001157, NP_{—}203127, NP_{—}031490, NP_{—}031491, AAK49776, AAK49777, CAB95312, NP_{—}001158, AAL32047, S68449, S68450, S68451, AAC50373, BAA85304, AAC52594, AAG41192, AAG41193, NP_{—}033818, AAB58376, XP_{—}066486, JC5964, AAG22971, AAD46161, AAG42316, AAG42316, AAC46988, AAC46988, NP_{—}477127, Q24307, O62640, S69545, S68452, AAB08398, AAK81891, AAF35319, AAF35320, NP_{—}035000, AAF35285, AAD56765, AAD56760, Q9R016, Q9JIB3, Q9JIB6, AAC32497, NP_{—}032696, AAF82752, Q9QWK5, AF135491, NP_{—}035002, AAD56759, AAL46972, AAK57560, NP_{—}046191, NP_{—}004527, A55478, NP_{—}064803, AAD00537, NP_{—}203195, AAF19819, AAC34373, NP_{—}148801, NP_{—}524101, AAF49548, NP_{—}037870, NP_{—}542729, NP_{—}075172, AAG37878, AAH14475, NP_{—}071444, AAC62261, NP_{—}031592, NP_{—}046197, NP_{—}057336, XP_{—}029485, NP_{—}059285, NP_{—}054056, NP_{—}203202, AAD45937, NP_{—}148900, NP_{—}047432, AAK81892, XP_{—}013050, AAG22969, AAL47170, AAL32047, S69545, AAC41610, AAC46988, AAL33679, NP_{—}524101, NP_{—}001157, AAB88044, Q90660, S68449, AAC83232, NP_{—}068520, NP_{—}031490, AAF35285, AAH11338, AAD56765, AAD56763, CAC36113, NP_{—}148878, NP_{—}048319, NP_{—}085041, XP_{—}038356, NP_{—}033819, AAL18251, AAL18250, AAG42494, AAK56308, NP_{—}071610, NP_{—}033819, AAG17540, AAL18251, NP_{—}001159, AAD26201, BAA93676, NP_{—}057336, NP_{—}071444, AAG37878, O14064, and AAC39171. Additionally, IAP polypeptides having at least 80%; desirably 90%; more desirably 95%; and most desirably 99% or greater sequence identify to one of the IAPs given above can be used in the methods of the invention. IAP polypeptides that can be used in the methods of the invention contain at least one BIR domain with at least 80%; desirably 85%; more desirably 95%; and most desirably 99% sequence identity to known BIR domains, e.g., the BIR domains disclosed in U.S. Pat. No. 6,156,535.

The following examples are meant to illustrate the invention. They are not meant to limit the invention in any way.

### EXAMPLE 1

### Expression of XIAP Protects Beta Islet Cells from Cytokine-Mediated Death

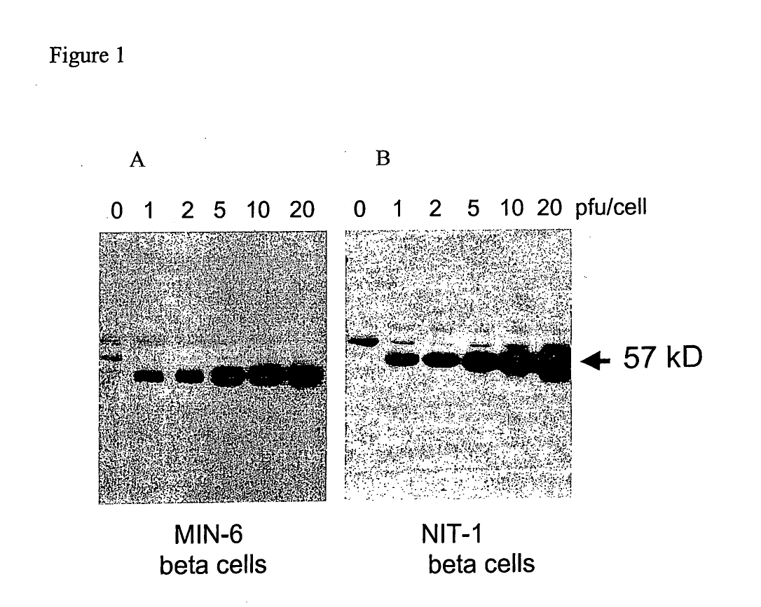

We tested whether XIAP expression in beta islet cells could provide protection from cytokine-mediated cell death using an in vitro tissue culture model system. Experiments indicated that XIAP could be effectively overexpressed in the transduced beta islet cell lines MIN-6 and NIT-1. We then transduced beta islet cells with 1, 2, 5, 10, or 20 pfu/cell of a recombinant adenovirus containing human XIAP cDNA (Ad-XIAP) to promote expression of human XIAP in these cell lines. Cell lysates were prepared and the level of XIAP expression was determined by western blot analysis. XIAP (˜57 kDa) was effectively expressed in MIN-6 (FIG. 1A) and NIT-1 (FIG. 1B) cells, with a maximum level of expression demonstrated using 5-20 pfu/cell (FIGS. 1A and 1B).

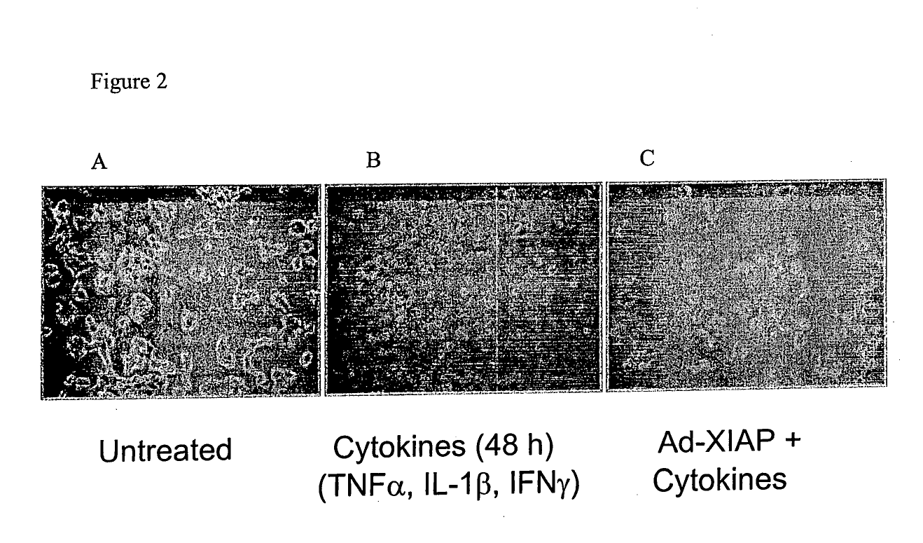

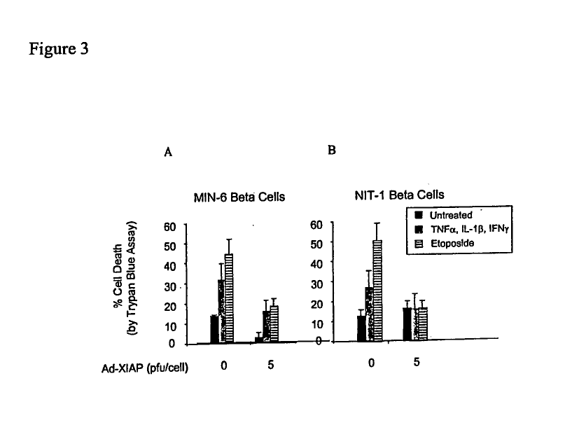

We next addressed whether expression of XIAP in beta islet cells could provide protection from cytokine-mediated cell death. ME-6 and NIT-1 cells were transduced (5 or 10 pfu/cell for 1 hour followed by 24 hour incubation) with Ad-XIAP or Ad-LacZ (as a control), then incubated for 24 hours in the presence of 2000 U/mL of tumor necrosis factor-α (TNF-α), interleukin-1β (IL-1β, and interferon-γ (INF-γ). Cell viability was then assessed by trypan blue exclusion. Death of MIN-6 cells following cytokine exposure was 32±8% vs. 13±1% in untreated cells (FIGS. 2A, 2B, and 3A). XIAP expression reduced cytokine-mediated cell death in MIN-6 cells to 16±6% (5 pfa/cell) and 22±4% (10 pfa/cell) similar to that observed in the presence of the pan-caspase inhibitor Boc-D-fmk (FIGS. 2C and 3B). XIAP expression also protected NIT-1 beta islet cells from cytokine-mediated death (25±8% to 6±4% at 10 pfu/cell and ˜15% at 5 pfu/cell; FIG. 3B). These data demonstrate that expression of MAP by recombinant adenovirus transduction protects transduced beta islet cells from cytokine-mediated cell death in vitro.

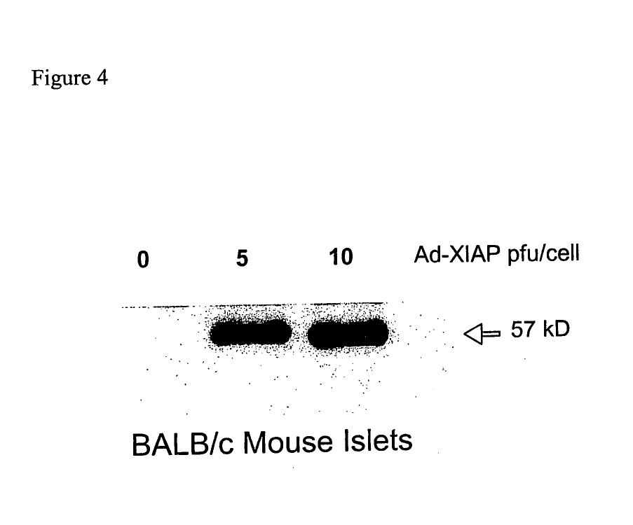

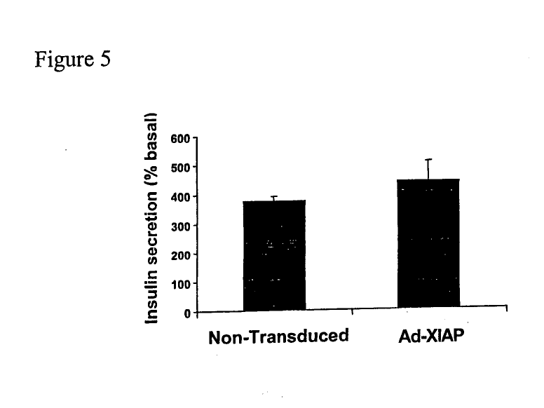

We next sought to demonstrate XIAP expression in isolated Balb/c mice islet cells prior to transplantation into allogeneic diabetic recipients. Isolated Balb/c mouse islet cells were transduced with Ad-MAP using 5 or 10 pfu/cell and incubated to allow expression. Lysates were prepared and examined by western blot analysis. XIAP expression was observed in isolated Balb/c mouse islet cells at both 5 pfu/cell and 10 pfu/cell (FIG. 4). We also determined that XIAP expression in Ad-XIAP transduced Balb/c mouse islet cells after 48 hours in culture does not impair beta islet cell function in vitro. Glucose-stimulated insulin secretion from non-transduced Balb/c mouse islet cells was equivalent to glucose-stimulated insulin secretion from Ad-MAP transduced Balb/c mouse islet cells (FIG. 5).

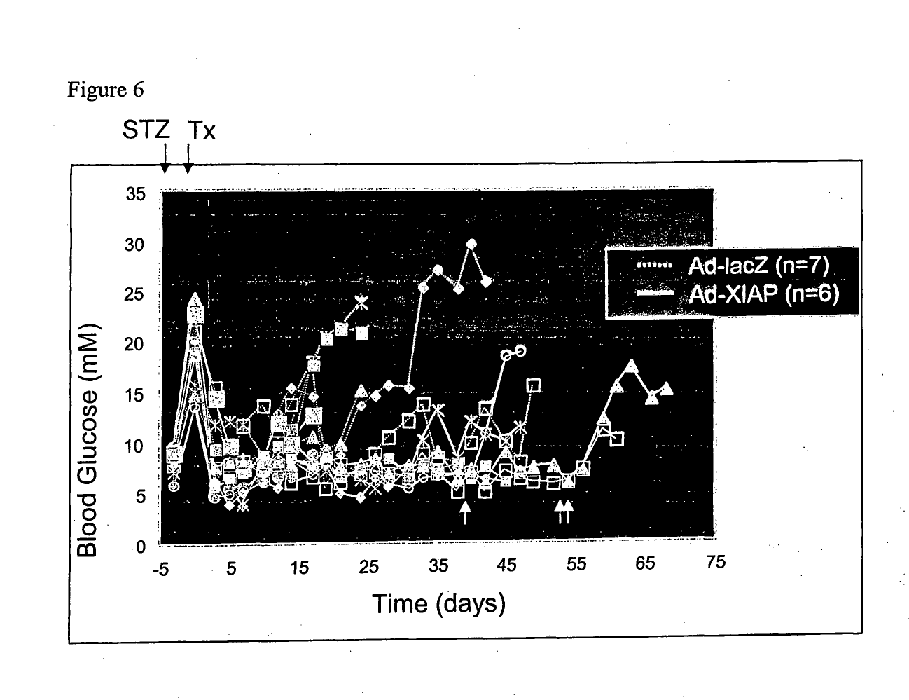

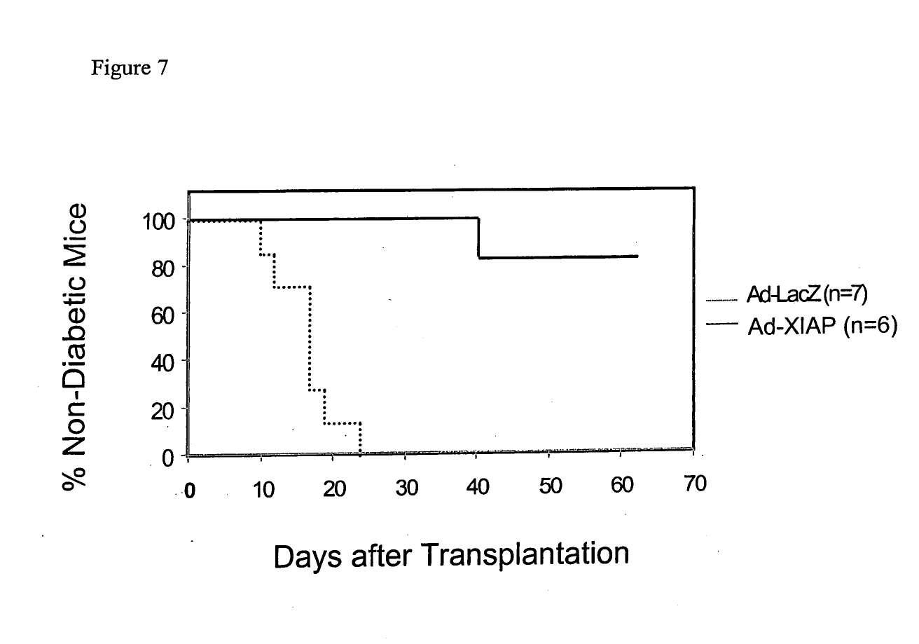

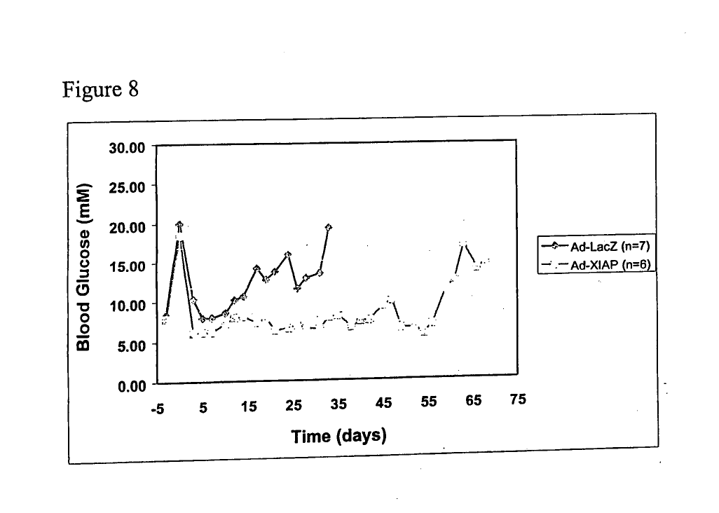

We then determined whether XIAP expression in islet cells prior to transplantation into allogeneic diabetic recipients would protect beta islet cells from immune-mediated cell death in vivo and enhance graft survival. Following overnight transduction with ˜10 pfu/cell Ad-XIAP (n=6) or Ad-LacZ (n=7; control group) 500 islet cells isolated from Balb/c mice were transplanted underneath the kidney capsule of CBA/J mice rendered diabetic by intraperitoneal injection of 325 mg/kg streptozotocin. Normoglycemia was maintained up to 62 days post-transplantation in 5 of 6 mice that received Ad-XIAP transduced islet cells (FIG. 6), whereas 6 of 7 mice that received Ad-LacZ were hyperglycemic (blood glucose≧11; mM; FIG. 6) by day 10 post-transplant and all mice in this group had rejected their islet graft by day 24 (FIGS. 7 and 8).

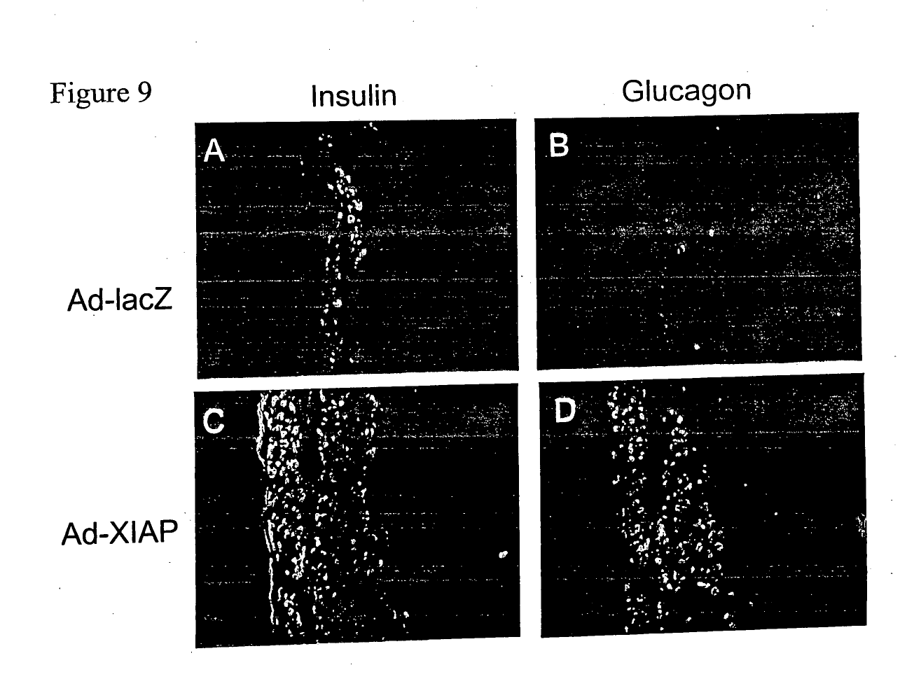

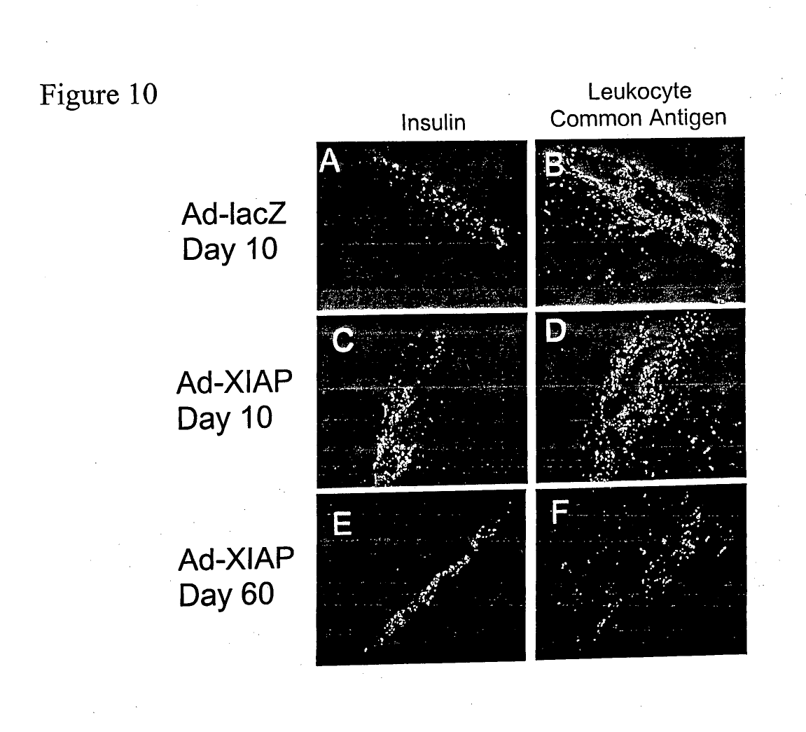

Histological analysis of beta islet cell allografts was also performed. We determined that XIAP expression resulted in the survival of beta islet cells (compare FIG. 9A with FIG. 9C) and glucagons-positive alpha cells (compare FIG. 9B with FIG. 9D). XIAP expression also led to immunotolerance in diabetic recipients of the islet allografts (compare FIG. 10A with FIGS. 10C and 10E, and FIG. 10B with FIGS. 10D and 10F). In summary, XIAP expression protects beta islet cells from cytokine-mediated cell death in vitro without loss of beta islet cell function, and provides long-lasting protection against islet allograft rejection in vivo.

### EXAMPLE 2

### Transplantation of Islet Cells into Diabetic Patients

Although new and more potent immunosuppressive agents are becoming available, many of these agents damage the beta islet cells or induce peripheral insulin resistance. To protect the cells prior to transplantation and to overcome the immunologic challenge, we provide transplantable beta islet cells transduced to express an IAP polypeptide. Expression of the IAP polypeptide increases the survival of the beta islet cells prior to transplantation as well as post transplantation, such that the cells can overcome immune-mediated and cytokine challenge, and remain viable for an extended period of time in the diabetic subject.

The following procedure for treating a diabetic patient using the methods of the invention are taken, in part, from Shapiro et al., New Engl. J. Med. 343:230-238 (2000) and similar methods can also be found in Contreras et al., Transplantation 71:1015-1023 (2001) and Guo et al., Cell Transplant 8:661-671 (1999).

The diabetic patient is first placed on an acceptable immunosuppression regimen, as determined by one skilled in the art, immediately before transplantation. Methods and prevention of graft rejection can be generally found in the art and as disclosed in U.S. Pat. No. 5,958,403.

Pancreatic beta islet cells are removed from brain-dead donors and stored in chilled University of Wisconsin preservation solution (Viaspan). Beta islet cells are isolated by perfusing the ducts in a controlled fashion with a cold enzyme (Liberase human islet, Roche). The islet cells are then separated by gentle mechanical dissociation and purified. The purified islet cells are cultured overnight with ˜10 pfu/cell recombinant adenovirus (Ad) or adeno-associated virus (AAV) containing XIAP cDNA operably linked to a human insulin promoter. Following an overnight incubation, the virus solution is removed and the islet cells are gently washed to remove any remaining virus. To minimize the risk of islet cell injury as a result of cold ischemia, the freshly prepared islet cells are transplanted immediately after transduction by the recombinant virus.

Islet cell preparations that have more than 4,000 islet cell equivalents per kilogram of the recipient's body weight in a packed-tissue volume of less than 10 mL are judged to be safe for transplantation. Each islet cell preparation is matched to the recipient's blood type and cross-matched for lymphocytotoxic antibodies, but no attempt at HLA matching need be made.

The subject is sedated and a percutaneous transhepatic approach is used to gain access to the portal vein for transplantation into the liver. Once access is confirmed, a catheter is placed within the main portal vein. The final islet preparation is suspended in 120 mL of medium 199 containing heparin and human albumin, and is infused over a period of five minutes.

Insulin therapy is discontinued after transplantation and is not resumed unless serum glucose concentrations rise above 200 mg per deciliter (11.1 mmol per liter). Serum glucose concentrations are monitored by memory capillary glucose meters, and the resulting data are analyzed. To determine the extent of fluctuations in glucose concentrations in the recipient, the mean amplitude of the differences in the major fluctuations in high and low glucose values during two 24-hour periods is determined. A minimum of seven measurements of capillary glucose are obtained (before a meal, two hours after a meal, at bedtime, and at 3 a.m.).

Successful transplantation results in a decrease in the insulin requirements of the subject. Serum is also analyzed for anti-insulin antibody and islet-cell antibody before and after transplantation. The lack of an increase in these parameters is generally due to a beneficial effect of immunosuppression.

The homeostatic model assessment is used to estimate insulin sensitivity by comparing fasting glucose and insulin data from the transplant recipients after they have achieved insulin dependence to normal subjects without diabetes.

The subject is also monitored for transplantation-related complications, such as cytomegalovirus infection and bleeding at the surgical site. The subject is also monitored for immunosuppressant-related conditions, lipid concentrations, and serum concentrations of creatine, cholesterol, and triglycerides.

### EXAMPLE 3

### Transplantation of Islet Cells into Diabetic Patients by Encapsulation

Human type 1 diabetic patients can also be administered XIAP-expressing insulin-producing cells that have been encapsulated. Encapsulation of cells is described in U.S. Pat. No. 6,303,355 and in Duvivier-Kali et al., Diabetes 50:1698-1705 (2001). Encapsulation of the insulin-producing cells (e.g., beta islet cells) protects the cells from a cellular reaction and toxic cytokines. Post-encapsulation, the cells, now residing in an immunoprotective barrier, can be implanted under the kidney capsule, in the liver, in the pancreas, or in the peritoneal cavity. Serum C-peptide production and blood glucose levels are monitored over several months to determine whether transplanted islet cells are producing insulin.

An amount of encapsulated islet cells to produce sufficient insulin to control glycemia in the subject is provided by any suitable means, including but not limited to surgical implantation and intraperitoneal injection. The International Islet Transplant Registry has recommended transplants of at least 6,000 islet cells, equivalent to 150 μm in size, per kilogram of recipient body weight, to achieve normoglycemia. However, it will be apparent to those skilled in the art that the quantity of microcapsules transplanted depends on the ability of the microcapsules to provide insulin in vivo, in response to glucose stimulation. One skilled in the art will be able to determine suitable transplantation quantities of microcapsules, using techniques that are known in the art.

### EXAMPLE 4

### In Vivo Treatment of Diabetic Patients with Recombinant Adenovirus or Adeno-associated Virus

Patients diagnosed with type 1 diabetes can be treated using in vivo methods consisting of administration of a recombinant adenovirus (Ad) or adeno-associated virus (AAV) containing a human IAP cDNA under the control of a promoter (e.g., a human insulin promoter) for expression in insulin-producing cells (e.g., beta islet cells). In vivo therapy involves transfection of an IAP nucleic acid directly into the cells of a recipient host without the need for prior removal of those cells from the recipient host.

In vivo delivery is desirably accomplished by (1) infusing an adenovirus or adeno-associated virus vector construct into a blood vessel that perfuses the pancreas or (2) injecting an adenovirus or adeno-associated virus vector construct directly into the pancreas. In an especially desired in vivo embodiment, a catheter is inserted into a blood vessel in the neck of an organism and the tip of the indwelling catheter is advanced with fluoroscopic guidance to a position in an artery that perfuses a portion of the pancreas. It is desired that the tip of an indwelling catheter be placed in proximity to an area of the pancreas that contains the cells to be transfected. The Ad or AAV can also be directly targeted to beta islet cells using beta islet cell-specific surface antigens, although this is not required. Adenovirus or adeno-associated virus is administered to patients desirably by means of intravenous administration in any suitable pharmacological composition, either as a bolus or as an infusion over a period of time. Injection of the recombinant virus directly into the pancreas, or into a blood vessel that perfuses the pancreas will promote incorporation of the human IAP cDNA into pancreatic cells (e.g., beta islet cells), which will provide protection against cytokine-mediated immune system challenge and will decrease or prevent immune system-mediated cell death of pancreatic insulin-producing cells.

After delivery of an adenovirus or adeno-associated virus vector construct to the cells of the pancreas, the cells are maintained under physiological conditions and for a period of time sufficient for the adenovirus or adeno-associated virus vector construct to infect the pancreas cells and for cellular expression of the IAP polypeptide contained in that construct.

Physiological conditions are those necessary for viability of the pancreas cells and include conditions of temperature, pH, osmolality and the like. In a desired embodiment, temperature is from about 20° C. to about 50° C., more desirably from about 30° C. to about 40° C. and, even more desirably about 37° C. pH is preferably from about a value of 6.0 to a value of about 8.0, more desirably from about a value of about 6.8 to a value of about 7.8 and, most desirably about 7.4. Osmolality is desirably from about 200 milliosmols per liter (mosm/L) to about 400 mosm/l and, more desirably from about 290 mosm/L to about 310 mosm/L. Other physiological conditions needed to sustain pancreas cell viability are well known in the art.

A time period sufficient for expression of an IAP polypeptide in a pancreas cell varies inter alia, as is well known in the art, on the type of adenovirus or adeno-associated virus vector used and the method of delivery. It should also be pointed out that because that the adenovirus or adeno-associated virus vector employed may be replication defective it may not be capable of replicating in the cells that are ultimately infected.

An adenovirus or adeno-associated virus vector construct is typically delivered in the form of a pharmacological composition that comprises a physiologically acceptable carrier and the adenovirus or adeno-associated virus vector construct. An effective amount of an adenovirus or an adeno-associated virus vector construct is delivered, and consists of 5 pfu/cell, 10 pfu/cell, or 20 pfu/cell, or any other amount that is effective for promoting expression of an IAP polypeptide in the target cells. Means for determining an effective amount of an adenovirus or an adeno-associated virus vector construct are well known in the art.

As is well known in the art, a specific dose level for any particular recipient depends upon a variety of factors including the infectivity of the adenovirus or adeno-associated virus vector, the age, body weight, general health, sex, diet, time of administration, route of administration, rate of excretion, and the severity of the diabetic condition of the recipient.

### EXAMPLE 5

### Preventative Early Detection and Treatment of Diabetes

Type 1 diabetes can be detected before a significant loss of beta islet cell function. As described in U.S. Pat. No. 6,300,089, hereby incorporated by reference, autoantibodies can be detected that signal the potential to develop diabetes type 1. These autoantibodies are present up to several years before the clinical manifestations of insulin-dependent diabetes (IDD) are observed. Early detection of these autoantibodies can allow preventative treatment by the methods of the invention. Providing a subject identified as likely to develop type 1 diabetes with beta islet cells transduced with an IAP will reduce the extent of beta islet cell apoptosis and the full clinical manifestations of type 1 diabetes.

### Other Embodiments

All publications and patents mentioned in this specification are herein incorporated by reference to the same extent as if each independent publication or patent application was specifically and individually indicated to be incorporated by reference.

What is claimed is:

1. A method of treating diabetes in a subject in need thereof, said method comprising:
providing in said subject one or more cells capable of producing insulin or one or more cells capable of producing progeny cells that produce insulin; wherein said cells express a trangene encoding an IAP polypeptide, or fragment thereof, under the control of a constitutive, inducible or cell-specific promoter that increases survival of said cells relative to untreated control cells not expressing said IAP.

2. The method of claim 1, wherein said cells are transduced ex vivo.

3. The method of claim 1, wherein said cells are transduced with a viral vector.

4-5. (canceled)

6. The method of claim 1, wherein said constitutive promoter is selected from the group consisting of the CMV promoter, the SV-40 promoter, and the actin promoter.

7. The method of claim 1, wherein said cell-specific or inducible promoter is a human insulin promoter.

8. (canceled)

9. The method of claim 1, wherein said subject is a human.

10. The method of claim 1, wherein said diabetes is type 1 diabetes.

11. The method of claim 1, wherein said nucleic acid sequence is selected from the group consisting of xiap, hiap-1, hiap-2, m-xiap, m-hiap-1, or m-hiap-2.

12. The method of claim 1, wherein said IAP polypeptide comprises at least one BIR domain and has caspase-inhibiting activity.

13. The method of claim 12, wherein said IAP polypeptide comprises two BIR domains.

14. The method of claim 1, wherein said cells comprise pancreatic beta islet cells.

15. The method of claim 1, wherein said cells comprise adult stem cells or embryonic stem cells that are capable of differentiating into insulin-producing cells or that are capable of producing progeny cells that are insulin-producing cells.

16. The method of claim 1, wherein said cells are isogeneic cells.

17. The method of claim 1, wherein said cells are allogeneic cells.

18. The method of claim 1, wherein said cells are xenogeneic cells.

19. The method of claim 18, wherein said xenogeneic cells are isolated from a pig, a sheep, or a baboon.

20. The method of claim 18, wherein said xenogeneic cells are genetically engineered to have increased survival in a recipient following transplantation, relative to a control cell not expressing said IAP.

21. The method of claim 1, wherein said transplanting is into the pancreas, the liver, or the kidney of said subject.

22. The method of claim 21, wherein said transplanting is into the kidney capsule of said kidney.

23. The method of claim 1, wherein said expression of said IAP polypeptide increases survival of said cells by at least 20%, relative to the survival of untreated control cells not expressing said IAP.

24-53. (canceled)

54. The method of claim 1, wherein said method further comprises administering an immunosuppressive agent to said subject.

55. The method of claim 54, wherein said immunosuppressive agent is selected from the group consisting of cyclosporin, cyclophosphamide, prednisone, dexamethasone, methotrexate, azathioprine, mycophenolate, thalidomide, FK-506, sirolimus, tacrolimus, daclizumab, and systemic steroids.

56. The method of claim 1, wherein said method further comprises administering an anti-apoptotic agent to said subject in an amount sufficient to inhibit apoptosis.

57-62. (canceled)

63. A cell capable of producing insulin or capable of producing progeny cells that produce insulin, wherein said cells express a transgene encoding an IAP polypeptide or fragment thereof, under the control of a constitutive, inducible, or cell-specific promoter, wherein expression of said IAP polypeptide increases survival of said cell relative to an untreated control cell not expressing said IAP.

64. The cell of claim 63, wherein said XIAP fragment comprises at least one BIR domain.

65. The cell of claim 64, wherein said BIR domain is a BIR3 domain.

66. The cell of claim 63, wherein said cell is a beta-islet cell.

67. The method of claim 1, wherein said cells are transduced in situ.

68. The method of claim 1, wherein said providing in said subject is by transplantation.

69. A medicament for the treatment of diabetes, said medicament comprising a cell capable of expressing insulin or capable of producing progeny cells that produce insulin in an amount sufficient to treat said diabetes, wherein said cell is transduced with a heterologous human XIAP polypeptide or fragment thereof under the control of a constitutive, inducible, or cell-specific promoter, wherein expression of said IAP polypeptide increases survival of said cell relative to an untreated control cell not expressing said IAP.
